(19) United States
(12) Patent Application Publication
(10) Pub. No.: US 2026/0220773 A1
(43) Pub. Date: Jul. 30, 2026

(54) DETECTING ATRIAL FIBRILLATION USING VISUALLY DISCERNIBLE FACIAL EXPESSIONS

(71) Applicant: Gill R. Tsouri, Rochester, NY (US)

(72) Inventor: Gill R. Tsouri, Rochester, NY (US)

(73) Assignee: Rochester Institute of Technology, Rochester, NY (US)

(21) Appl. No.: 19/146,061

(22) PCT Filed: Jan. 5, 2024
(86) PCT No.: PCT/US2024/010521

§ 371 Date: Jul. 7, 2025

Related U.S. Application Data
(60) Provisional application No. 63/437,155, filed on Jan. 5, 2023.

(51) Int. Cl. G06T 7/00; A61B 5/103; G06V 10/764; G06V 10/774; G06V 40/16

(52) U.S. Cl. CPC G06T 7/0012; G06V 10/764; G06V 10/774; G06V 40/171; G06V 40/174; A61B 5/1032; G06T 2207/20081; G06T 2207/30201; G06V 2201/03

(57) ABSTRACT

A method and device for detecting Atrial Fibrillation (AF), includes acquiring an image of the face of a subject; extracting facial expressions from the image; and determining the presence or absence of AF by applying the extracted facial expressions to a classifier trained to detect AF. The method can include training a subject specific or generic classifier to determine the presence or absence of AF based on based the subject's facial expressions. The subject can be prompted to actively take a measurement using an FDA-approved AF-detection device when the presence of AF is detected.

### CROSS REFERENCE

This application claims the benefit of the filing date of U.S. Provisional Patent Application No. 63/437,155, filed Jan. 5, 2023, which is hereby incorporated by reference in its entirety.

### FIELD

The present disclosure relates to detecting Atrial Fibrillation (AF) from visually discernable facial expressions, and in particular to detecting AF from visually discernable facial expressions obtained while monitoring the subject.

### BACKGROUND

Long-term monitoring away from the hospital can be done by providing the patient with a device to continuously monitor AF recurrence, AF burden and heart rate. Devices that continuously monitor AF and HR are typically expensive and are attached to the skin (ECG patch, apple watch, etc.), they have a limited lifespan, require maintenance and irritate the skin. Other devices that are not attached to the skin require active participation by the subject (e.g., placing fingers on electrodes, etc.). Long term monitoring using both types of devices suffer from a low adherence problem. Namely, the tendency of subjects not to comply with monitoring procedures over long periods of time. Patients would remove an ECG patch due to skin irritation, neglect charging devices and tend to avoid taking active measurements when they feel well.

Currently, the monitoring of AF patients is limited to infrequent visits to the hospital. A typical treatment procedure would be to subscribe heart rate regulation drugs at a certain dosage with titration performed across lengthy periods between visits or if the patient's condition is deteriorating. Patient treatment would benefit greatly from long-term monitoring at home away from the hospital.

The prior art fails to disclose methods for detecting AF based on facial expressions. Rather, public disclosures relate to using facial expressions to detect other conditions such as stroke and Parkinson. A list of these publications includes Ali, Mohammad Rafayet, et al. “Facial expressions can detect Parkinson's disease: Preliminary evidence from videos collected online.” NPJ digital medicine 4.1(2021 ): 1-4; Abbott, Jacenta D., et al. “The perception of positive and negative facial expressions by unilateral stroke patients.” Brain and cognition 86 (2014): 42-54; Heponiemi, Tarja, et al. “Experiencing positive affect and negative affect during stress: Relationships to cardiac reactivity and to facial expressions.” Scandinavian Journal of Psychology 47.5 (2006 ): 327-337; and Marshall, Amanda C., et al. “Cardiac interoceptive learning is modulated by emotional valence perceived from facial expressions.” Social cognitive and affective neuroscience 13.7 (2018 ): 677-686.

### SUMMARY

In accordance with one aspect of the present disclosure, there is provided a method for detecting Atrial Fibrillation (AF), including:
acquiring an image of the face of a subject;
extracting facial expressions from the image; and
determining the presence or absence of AF by applying the extracted facial expressions to a classifier trained to detect AF.

In accordance with another aspect of the present disclosure, there is provided a method for detecting Atrial Fibrillation (AF), including:
acquiring an image of the face of a subject;
extracting facial expressions from the image;
determining the presence or absence of AF by applying the extracted facial expressions to a classifier trained to detect AF; and
prompting the subject to actively take a measurement using an FDA-approved AF-detection device when the presence of AF is detected.

In accordance with another aspect of the present disclosure, there is provided a method for detecting Atrial Fibrillation (AF), including:
acquiring an image of the face of a subject;
extracting facial expressions from the image; and
determining the presence or absence of AF by applying the extracted facial expressions to a classifier trained to detect AF, which includes detecting facial landmarks from the image of the subject,
using the facial landmarks to extract Action Units (AUS) using the Facial Action Code System (FACS),
feeding the extracted AUs to the trained classifier, and
determining the presence or absence of AF in the subject based on an output from the trained classifier.

In accordance with another aspect of the present disclosure, there is provided a method for detecting Atrial Fibrillation (AF), including:
acquiring an image of the face of a subject;
extracting facial expressions from the image;
determining the presence or absence of AF by applying the extracted facial expressions to a classifier trained to detect AF, which includes detecting facial landmarks from the image of the subject,
using the facial landmarks to extract Action Units (AUs) using the Facial Action Code System (FACS),
feeding the extracted AUs to the trained classifier, and
determining the presence or absence of AF in the subject based on an output from the trained classifier; and
prompting the subject to actively take a measurement using an FDA-approved AF-detection device when the presence of AF is detected.

In accordance with another aspect of the present disclosure, there is provided a method for detecting Atrial Fibrillation (AF), including:
acquiring an image of the face of a subject;
extracting facial expressions from the image; and
determining the presence or absence of AF by applying the extracted facial expressions to a classifier trained to detect AF, wherein training the classifier includes:
providing the subject with an FDA-approved AF-detection device that continuously monitors the subject,
obtaining a single image or multiple images or sequence of images containing facial expressions of the subject,
determining a ground truth for the presence of AF in the subject based on the FDA-approved AF-detection device,
labeling the obtained image or images of the subject as “AF” or “non-AF” based on the ground truth, and
providing a trained subject specific classifier by training the classifier to detect AF based the subject's facial expressions in the labelled image or images.

In accordance with another aspect of the present disclosure, there is provided a method for detecting Atrial Fibrillation (AF), including:
acquiring an image of the face of a subject;
extracting facial expressions from the image;
determining the presence or absence of AF by applying the extracted facial expressions to a classifier trained to detect AF, wherein training the classifier includes:
providing the subject with an FDA-approved AF-detection device that continuously monitors the subject,
obtaining a single image or multiple images or sequence of images containing facial expressions of the subject,
determining a ground truth for the presence of AF in the subject based on the FDA-approved AF-detection device,
labeling the obtained image or images of the subject as “AF” or “non-AF” based on the ground truth, and
providing a trained subject specific classifier by training the classifier to detect AF based the subject's facial expressions in the labelled image or images; and
prompting the subject to actively take a measurement using an FDA-approved AF-detection device when the presence of AF is detected.

In accordance with another aspect of the present disclosure, there is provided a method for detecting Atrial Fibrillation (AF), including:
acquiring an image of the face of a subject;
extracting facial expressions from the image; and
determining the presence or absence of AF by applying the extracted facial expressions to a classifier trained to detect AF, wherein training the classifier includes:
providing a plurality of subjects with an FDA-approved AF-detection device that continuously monitors the subjects,
obtaining a single image or multiple images or sequence of images containing facial expressions of the subjects,
determining a ground truth for the presence of AF in the subject based on the FDA-approved AF-detection device,
labeling the obtained image or images of the subjects as “AF” or “non-AF” based on the ground truth, and
providing a trained generic classifier by training the classifier to detect AF based the plurality of subject's facial expressions in the labelled image or images.

In accordance with another aspect of the present disclosure, there is provided a method for detecting Atrial Fibrillation (AF), including:
acquiring an image of the face of a subject;
extracting facial expressions from the image; and
determining the presence or absence of AF by applying the extracted facial expressions to a classifier trained to detect AF, wherein training the classifier includes:
providing a plurality of subjects with an FDA-approved AF-detection device that continuously monitors the subjects,
obtaining a single image or multiple images or sequence of images containing facial expressions of the subjects,
determining a ground truth for the presence of AF in the subject based on the FDA-approved AF-detection device,
labeling the obtained image or images of the subjects as “AF” or “non-AF” based on the ground truth, and
providing a trained generic classifier by training the classifier to detect AF based the plurality of subject's facial expressions in the labelled image or images; and
prompting the subject to actively take a measurement using an FDA-approved AF-detection device when the presence of AF is detected.

In accordance with another aspect of the present disclosure, there is provided a device for detecting Atrial Fibrillation (AF), including:
computer readable program instructions for:
   acquiring an image of the face of a subject;
   extracting facial expressions from the image; and
determining the presence or absence of AF by applying the extracted facial expressions to a classifier trained to detect AF.

These and other aspects of the present disclosure will become apparent upon a review of the following detailed description and the claims appended thereto.

### BRIEF DESCRIPTION OF THE DRAWINGS

FIG. 1 is a graph of the frequency of appearance of each AU in all images compared to its appearance in images labeled as AF (left) and pictures of representative facial expression corresponding to each AU (right);

FIG. 2 presents boxplots per AU for AF and non-AF images in the dataset;

FIG. 3 is a graph of Receiver Operating Characteristic (ROC) curves obtained for binary subject-generic classifiers; and

FIG. 4 is a graph of Receiver Operating Characteristic (ROC) curves obtained for a binary subject-specific classifier.

### DETAILED DESCRIPTION

The disclosure can be used to perform efficient long-term monitoring of AF patients away from the hospital. This can potentially be expanded to perform efficient long-term monitoring of other cardiac conditions.

The disclosure can be used to monitor AF in the background using any smart device enabled with embedded front-facing camera, e.g., smartphones, tablets and laptops. Essentially, the disclosure leverages existing screen time on existing personal devices to perform passive monitoring. It follows that the disclosure supports long-term monitoring without relying on subject participation with monitoring procedures and without requiring a dedicated costly device. In addition, since the disclosure can be embodied and implemented using software, a simple download of an APP can be used to distribute the monitoring service, thereby making the service accessible to anyone in the world and for a fraction of the distribution costs associated with existing solutions.

In an embodiment, a method for detecting Atrial Fibrillation (AF) includes the following:
(i) acquiring an image of the face of a subject;
(ii) extracting facial expressions from the image; and
(iii) determining the presence or absence of AF by applying the extracted facial expressions to a classifier trained to detect AF.

In a further embodiment, when the presence of AF is detected by the present disclosure the subject can be prompted to actively take a measurement using an FDA-approved AF-detection device to confirm the presence of AF.

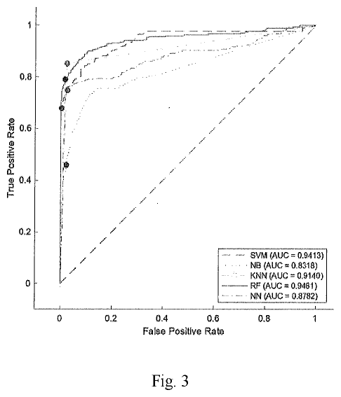

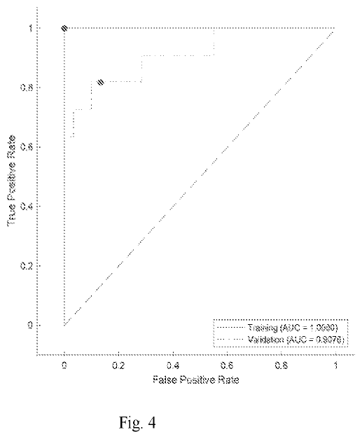

In an embodiment, a machine learning or deep learning classifier can be applied where the classifier's output is a number indicating the level of confidence in the determination of the presence of AF, e.g., a number between 0 and 1, where 0 represents complete certainty of no AF, 1 represents complete certainty of AF and any number between 0 and 1 represents uncertainty. Such classifiers can be implemented using classification architectures known to a person skilled in the art. In such embodiments, a threshold is applied to the classifier output resulting in a binary determination (AF when output is above the threshold and not AF if not). The value of the threshold is found by generating a Receiver Operating Characteristic (ROC) by scanning through the range of the threshold values and evaluating the True Positive Rate (TPR) and False Positive Rate (FPR) per threshold. Examples of ROC curves are presented in FIG. 3 and FIG. 4.

In an embodiment, the extracted facial expressions can be applied to a classifier trained to detect AF to determine the presence or absence of AF by:
(i) detecting facial landmarks from the image of the subject;
(ii) using the facial landmarks to extract Action Units (AUs) using the Facial Action Code System (FACS);
(iii) feeding the extracted AUs to the trained classifier; and
(iv) determining the presence or absence of AF in the subject based on an output from the trained classifier.

An example of applying the extracted facial expressions to a classifier trained to detect AF includes the following steps:
(i) Acquire an image of the face of a subject;
(ii) Detect 64 basic facial landmarks of the subject;
(iii) Using the landmarks, detect the presence of Action Units (AUs) using the Facial Action Code System (FACS);
(iv) Feed the binary indications for AUs to a binary machine learning classifier, e.g., Random Forest and Support Vector Machine; and
(v) Determine the presence (or absence) of AF based on the classifier output.

In an embodiment, training a subject specific classifier includes the following:
(i) providing the subject with an FDA-approved AF-detection device that continuously monitors the subject;
(ii) obtaining a single image or multiple images or sequence of images containing facial expressions of the subject;
(iii) determining a ground truth for the presence of AF in the subject based on the FDA-approved AF-detection device;
(iv) labeling the obtained image or images of the subject as “AF” or “non-AF” based on the ground truth; and
(v) providing a trained subject specific classifier by training the classifier to detect AF based the subject's facial expressions in the labelled image or images.

The following is an example of a process for deploying and using a camera-based classifier for inferring the presence of AF by identifying specific facial expressions in images captured by the camera. Namely, specific facial expressions or combinations of facial expressions can be associated with the presence of AF and other specific facial expressions or combinations of facial expressions can be associated with the absence of AF. In this example, the classifier can be designed to tailor for a specific subject being monitored and can be composed of two phases: (a) training phase for fine tuning the classifier's parameters for the specific subject and (b) application phase during which the classifier's parameters are fixed and the classifier is used to detect AF in the specific subject.

The training phase includes the following steps:
(i) Download software to a device to be used by the subject. The device needs to have a front-facing camera. The software would implement a classifier capable of detecting AF by analyzing a facial image with default parameter values;
(ii) Provide the subject with an FDA-approved AF-detection device that continuously monitors the subject, e.g., ECG patch;
(iii) Occasionally take a single (or multiple) photo (or sequence of pictures) of the subject;
(iv) Determine ground truth for the presence of AF based on the FDA-approved device;
(v) Label each picture of the subject as “AF” or “non-AF” based on the ground truth by matching time of image capture with time of ECG-approved device capture; and
(vi) Use collected photos with their corresponding ground truth label to train the classifier to detect AF based on AFs collective impact on the subject's facial expressions.
   The application phase includes the following steps:
(i) Lock the trained subject specific classifier's parameters; and
(ii) Use the downloaded software and trained subject specific classifier to detect AF using subsequent images of the subject taken by the camera.
In this example the software and classifier are utilized to entirely replace the FDA-approved device to detect AF after training with the device is completed.

In an embodiment, training a generic classifier includes the following:
(i) providing a plurality of subjects with an FDA-approved AF-detection device that continuously monitors the subjects;
(ii) obtaining a single image or multiple images or sequence of images containing facial expressions of the subjects;
(iii) determining a ground truth for the presence of AF in the subject based on the FDA-approved AF-detection device;
(iv) labeling the obtained image or images of the subjects as “AF” or “non-AF” based on the ground truth; and
(v) providing a trained generic classifier by training the classifier to detect AF based the plurality of subject's facial expressions in the labelled image or images.

The following is an example of a generic classifier capable of detecting AF for any subject. In this example, a training phase includes the following steps:
(i) Conduct a large clinical study where software is downloaded to a device to be used by each subject. The device needs to have a front-facing camera. The software would capture images of the subject's face in the background;
(ii) Provide the subject with an FDA-approved AF-detection device that continuously monitors the subject, e.g., ECG patch;
(iii) Occasionally take a single (or multiple) photo (or sequence of pictures) of the subject;
(iv) Determine ground truth for the presence of AF based on the FDA-approved device;
(v) Label each picture of the subject as “AF” or “non-AF” based on the ground truth by matching time of image capture with time of ECG-approved device capture; and
(vi) Use collected photos from all subjects participating in the study with their corresponding ground truth label to train a classifier to detect AF based on AFs collective impact on the clinical study population's facial expressions.
   An application phase includes the following steps:
(i) Create software capable of capturing images using a front camera; and
(ii) Download the software to a device with a front facing camera and use it by applying the trained generic classifier to captured images to detect AF in a subject.

An example of confirming AF with an FDA-approved AF-detection device when AF is indicated includes the following steps:
(i) Download software to a device to be used by the subject. The device needs to have a front-facing camera. The software would implement a classifier (either subject specific or generic trained) capable of detecting AF by analyzing a facial image with default parameters values;
(ii) Provide the subject with an FDA-approved AF-detection device that requires active participation of the subject in the measurement, e.g., the Kardia Mobile Card;
(iii) Occasionally take a single (or multiple) photo (or sequence of pictures) of the subject and determine the presence of AF using the trained classifier; and
(iv) If AF is detected, prompt subject to actively take a measurement using the FDA-approved device.

In an embodiment of the confirmation process, the software and classifier act as an easy-to-use screening tool to help address the low adherence of subjects with prescribed long-term monitoring procedures. This benefit is amplified when by having the software operate in the background while the subject is making regular use of the device with front-facing camera, e.g., reading emails, watching videos, and surfing the web. So that initial screening is performed passively without requiring subject participation and action from the subject is required only occasionally to verify the presence of AF when indicated by the software.

As noted above, the disclosure has the following unexpected features and advantages:
(1) Requires no costly dedicated device.
(2) Leverages existing screen time of subjects using their own personal smart devices.
(3) Provides easy distribution of monitoring service via a software download.
(4) Provides contactless monitoring.
(5) Significantly alleviates the need for patient adherence with self-monitoring procedures.

Another important advantage of the present disclosure relates to signal integrity. All prior devices and methods rely on a lengthy measurement with a minimum duration of 30 seconds to provide an assessment of AF. Long duration capture exposes these methods to distortions in the signal due to motion of the subject and other environmental conditions. In contradistinction, the present disclosure can rely on snapshots of the face taken at an instance of time, thereby limiting the impact of motion on detection of AF.

The disclosure can be the basis for a downloadable APP (Android, iOS, Windows) to be subscribed by a physician to a patient diagnosed with AF. In this sense, the APP would be considered software as a medical device.

The disclosure can be used by insurance companies to reduce hospital readmissions of AF patients under coverage.

The disclosure can be used by pharma companies as a supplemental service in perform real time drug titration when prescribing rate regulation medication such as beta blockers.

The above-described embodiments of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, blockchain, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

The disclosure will be further illustrated with reference to the following specific examples. It is understood that these examples are given by way of illustration and are not meant to limit the disclosure or the claims to follow.

Example 1: Training a classifier with facial expressions from multiple individuals to identify AF and applying the generically trained classifier to subsequent facial expressions of a subject to detect AF.

FACS were introduced by Ekman in 1978 as a standardized coding of facial expressions. In FACS, facial expressions are broken down into individual muscle movements of the face called Action Units (AUS).

A set of images were acquired during a test of the face recognition functionality performed in a study relating to AF detection using video plethysmography. The data obtained from this study had AF detection ground truth based on 30 seconds time-based signal measurement of skin color changes. The images taken for the purpose of testing face recognition were associated with the ground truth for AF. This provided a database of facial images labelled with AF. The results below are based on that database.

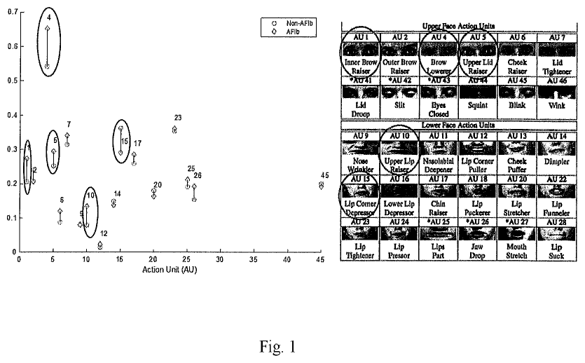

The prevalence of specific AUs in the presence of AFib to their overall prevalence suggest a relationship. Results are presented in FIG. 1 with the frequency of each AU (left) and the facial expression feature corresponding to each AU (right). The circles indicate AUs that diverge the most between Afib and overall. It is apparent that AUs are affected by the onset of AFib, suggesting that provided enough data is gathered, it should be possible to train a classifier to detect AFib using FACS.

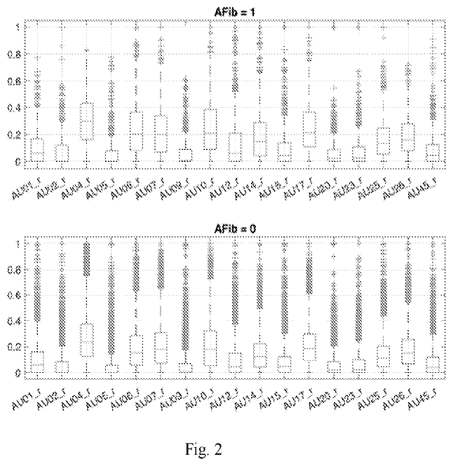

A deeper statistical analysis of the feature space provides the distribution of AUs during AF versus non-AF shown in FIG. 2. FIG. 2 presents boxplots per AU for AF and non-AF images in the dataset. In general, across all the AUs, the statistical properties (median, first quartile, third quartile, interquartile range) vary slightly. It is clear that different combinations of AUs are present during AF versus non-AF. As a single isolated AU observation, note the appearance of AU4 (Brow Lowered).

When attempting to train and validate subject-generic Machine Learning classifiers using the AUs as features, the Receiver Operating Characteristic (ROC) curves are obtained as shown in FIG. 3. FIG. 3 is a graph of Receiver Operating Characteristic (ROC) curves obtained for binary classifiers based on various classification architectures (SVM, NB, KNN, RF, NN) trained using all subjects data, i.e., subject-generic. These curves are based on the worst validation results following a 3-fold training and cross validation approach. Note that the Random Forest (RF) classifier provides the best performance followed closely by the Support Vector Machine (SVM) classifier.

Results suggest that a classifier can be designed for AF detection based on AUs facile features with a TPR of 0.9 and FPR of 0.1. These are encouraging results yet more data may be required to achieve higher validation accuracy and to validate the specific Processes (use cases) described in the disclosure.

Example 2: Training a classifier with patient-specific facial expressions to identify AF and applying the trained subject-specific classifier to subsequent facial expressions of the subject to detect AF.

A subject-specific classifier is expected to perform more accurately than a generic classifier, since the subject-specific classifier can be trained to identify the AFib expression of a particular subject. For example, utilizing a specific subject data, including 71 AFib samples and 284 non-AFib samples (355 samples in total), we trained a Fine-Tuned Random Forest (FT-RF) model to classify AFib based on that subject's specific facial features. FIG. 4 is a graph of Receiver Operating Characteristic (ROC) curves obtained for a binary classifier based on the Fine-Tuned Random Forest (FT-RF) architecture trained using data from a specific subject, i.e., subject-specific. FIG. 4 presents the performance of the classifier using 5-fold cross-validation. The worst (lowest AUC) cross-validated model of FT-RF is shown. Note that the validated classifier can provide True Positive Rate (aka Sensitivity) and False Positive Rate (aka 1-Specificity) of 0.8 simultaneously.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

What is claimed

1. A method for detecting Atrial Fibrillation (AF), comprising:
acquiring an image of the face of a subject;
extracting facial expressions from the image; and
determining the presence or absence of AF by applying the extracted facial expressions to a classifier trained to detect AF.

2. The method of claim 1, further comprising prompting the subject to actively take a measurement using an FDA-approved AF-detection device when the presence of AF is detected.

3. The method according to claim 1, wherein applying the extracted facial expressions comprises:
detecting facial landmarks from the image of the subject;
using the facial landmarks to extract Action Units (AUs) using the Facial Action Code System (FACS);
feeding the extracted AUs to the trained classifier; and
determining the presence or absence of AF in the subject based on an output from the trained classifier.

4. The method according to claim 1, wherein training the classifier comprises:
providing the subject with an FDA-approved AF-detection device that continuously monitors the subject;
obtaining a single image or multiple images or sequence of images containing facial expressions of the subject;
determining a ground truth for the presence of AF in the subject based on the FDA-approved AF-detection device;
labeling the obtained image or images of the subject as “AF” or “non-AF” based on the ground truth; and
providing a trained subject specific classifier by training the classifier to detect AF based the subject's facial expressions in the labelled image or images.

5. The method according to claim 1, wherein training the classifier comprises:
providing a plurality of subjects with an FDA-approved AF-detection device that continuously monitors the subjects;
obtaining a single image or multiple images or sequence of images containing facial expressions of the subjects;
determining a ground truth for the presence of AF in the subject based on the FDA-approved AF-detection device;
labeling the obtained image or images of the subjects as “AF” or “non-AF” based on the ground truth; and
providing a trained generic classifier by training the classifier to detect AF based the plurality of subject's facial expressions in the labelled image or images.

6. The method of claim 3, further comprising prompting the subject to actively take a measurement using an FDA-approved AF-detection device when the presence of AF is detected.

7. The method of claim 4, further comprising prompting the subject to actively take a measurement using an FDA-approved AF-detection device when the presence of AF is detected.

8. The method of claim 5, further comprising prompting the subject to actively take a measurement using an FDA-approved AF-detection device when the presence of AF is detected.

9. A device for detecting Atrial Fibrillation (AF), comprising:
computer readable program instructions for:
acquiring an image of the face of a subject;
extracting facial expressions from the image; and
determining the presence or absence of AF by applying the extracted facial expressions to a classifier trained to detect AF.
